(19) United States
(12) Patent Application Publication
(10) Pub. No.: US 2022/0333207 A1
(43) Pub. Date: Oct. 20, 2022

(54) PCR PRIMER COMPOSITION FOR DETECTING MUTATION OF TELOMERASE REVERSE TRANSCRIPTASE, AND USE THEREOF

(71) Applicant: GENINUS INC., Songpa-gu, Seoul (KR)

(72) Inventors: Kyoung-Mee KIM, Gangnam-gu, Seoul (KR); So Young KANG, Gangnam-gu, Seoul (KR)

(21) Appl. No.: 17/638,609

(22) PCT Filed: Aug. 26, 2020
(86) PCT No.: PCT/KR2020/011395

§ 371 Date: Feb. 25, 2022

(30) Foreign Application Priority Data
Aug. 30, 2019 (KR) 10-2019-0107643

(51) Int. Cl. C12Q 1/6886

(52) U.S. Cl. CPC C12Q 1/6886; C12Q 2600/156; C12Q 1/686

(57) ABSTRACT

The present invention relates to a PCR primer composition for detecting a mutation of a telomerase reverse transcriptase (TERT), and a use thereof, wherein the TERT gene may be amplified under specific conditions by using a forward primer set represented by SEQ ID NO: 1 and a reverse primer set represented by SEQ ID NO: 2, and thus a mutation of TERT may be detected.

### TECHNICAL FIELD

The present application claims priority for Korean patent application No. 10-2019-0107643 filed on Aug. 30, 2019, the entire description of which is a reference for the present application.

The present invention relates to a PCR primer composition for detecting a mutation of a telomerase reverse transcriptase, and a use thereof.

### BACKGROUND ART

Telomeres are terminal structures of eukaryotic chromosomes and have repetitive oligonucleotide sequences. Telomeres prevent chromosomes from damage or merging onto other chromosomes, and the length of a telomere is shortened at each cell division. After a certain number of cell division, the telomere length is extremely shortened to the extent in which the cell stops dividing and dies. Cells other than somatic cells, such as germ cells and cancer cells, produce telomerase that prevents shortening of telomeres, which enables unlimited proliferation of these cells. In specific diseases, the telomeric end is abnormally rapidly lost and leads to aging or degeneration of immature cells.

Telomerase reverse transcriptase (TERT) is a protein that affects cancer cell proliferation, and cancer may be diagnosed by examining its protein mutant genome. TERT is known as an accurate indicator of predicting the recurrence risk of papillary thyroid cancer, and the prognosis and the risk of metastasis in some brain tumor patients may be predicted using TERT. However, TERT is difficult to use in practice due to its inherent characteristics. In particular, the genome must be amplified to perform a genetic test, but TERT is inefficient because of its characteristics. Therefore, it is needed to study a method that may effectively amplify the TERT protein mutant genome. cl DESCRIPTION OF EMBODIMENTS

### Technical Problem

According to an embodiment, provided is a polymerase chain reaction (PCR) primer composition for a mutation of a telomerase reverse transcriptase (TERT), the PCR primer composition including a primer set represented by SEQ ID NO: 1 and a primer set represented by SEQ ID NO: 2.

According to another embodiment, provided is a kit for detecting a mutation of a TERT, the kit including the PCR primer composition.

According to another embodiment, provided is a method of detecting a mutation of a TERT, the method including mixing a nucleic acid template to be amplified to the PCR primer composition to prepare a mixture; performing an amplification reaction on the mixture; and detecting a mutation of a TERT in a product of the amplification reaction.

### Solution to Problem

According to an embodiment, provided is a polymerase chain reaction (PCR) primer composition for a mutation of a telomerase reverse transcriptase (TERT), the PCR primer composition including a primer set represented by SEQ ID NO: 1 and a primer set represented by SEQ ID NO: 2.

The mutation occurs in a promoter of a TERT gene and, more particularly, may refer to substitution of T for C, which is the 124th nucleotide of a gene represented by SEQ ID NO: 3; and/or substitution of T for C, which is the 146th nucleotide of a gene represented by SEQ ID NO: 3.

In one embodiment, the composition may include a reaction buffer solution, dNTPs, or a DNA polymerase. As the reaction buffer solution, a conventional PCR buffer solution including components such as Tris-HCl, KCl, (NH₄)₂SO₄, MgSO₄, or MgCl₂ may be properly modified and used. The 4 different dNTPs refer to dATP, dTTP, dGTP, and dCTP, and the DNA polymerase is not limited to a specific enzyme. Also, concentrations of the primers for PCR amplification may be in a range of 1 pmole to 50 pmole in a 20-μL PCR reaction solution, and the primer concentration may be determined by those skilled in the art. In one embodiment, the composition may further include a dye and/or a stabilizer to promote the convenience in experiments, the prevention of contamination by PCR product, and the improvement of stabilization and reactivity of DNA polymerase and dNTPs. Here, the dye may be at least one selected from bromophenol blue, xylene cyanole, bromocresol red, and cresol red, and the stabilizer may be at least one selected from gelatin, bovine serum albumin, Thesit, PEG-8000, and polyol. An amount of the dye in the composition may be in a range of 0.0001 weight % to 0.01 weight %, 0.001 weight % to 0.005 weight %, or 0.001 weight % to 0.003 weight %, based on the final composition. Also, a concentration of the stabilizer in the composition may be in a range of 2 mM to 1,000 mM, 100 mM to 500 mM, or 100 mM to 300 mM in the final composition.

According to another embodiment, provided is a kit for detecting a mutation of a TERT, the kit including the PCR primer composition. Details of the PCR primer composition are the same as described above. The kit may detect a mutation of a TERT and thus may predict the risk of recurrence of papillary thyroid cancer, thereby predicting the prognosis and the risk of metastasis of brain tumor in some patients. In some embodiments, the kit may be a reverse transcription polymerase chain reaction (RT-PCR) kit, a DNA chip kit, an enzyme-linked immunosorbent assay (ELISA) kit, a protein chip kit, a rapid kit, or a multiple reaction monitoring (MRM) kit. Also, the kit for detecting a mutation of a TERT may further include one or more component compositions, solutions, or apparatuses appropriate for an analysis method. For example, the kit may be a kit including essential elements necessary to perform RT-PCR. The RT-PCR kit includes primer pairs, each specific to the mutant gene. The primer is a nucleotide having a sequence specific to the nucleic acid sequence of each gene, which is approximately from 7 base pairs (bp) to 50 bp in length, and more preferably, approximately from 10 bp to 30 bp in length. The primer may also include a primer specific to the nucleic acid sequence of a control group gene. Additionally, the RT-PCR kit may include test tube or other appropriate container, reaction buffer solution (with varying pH and magnesium concentration), deoxynucleotide (dNTPs), enzyme such as Taq-polymerase and RT-PCR enzyme, DNAse, DNAse inhibitor, DEPC-water, sterile water, etc. Also, for example, the DNA chip kit may include a substrate to which gene or cDNA or oligonucleotide corresponding to a fragment thereof is attached, and a reagent, agent, enzyme, etc. to prepare fluorescent-labeled probe. The substrate may include a control group gene or cDNA or oligonucleotide corresponding to a fragment thereof. Further, for example, the kit may include an essential element necessary to carry out ELISA. The ELISA kit may include an antibody specific to a protein. The antibody has high specificity and affinity to each marker protein, and almost no cross reactivity to the other proteins, and is a monoclonal, polyclonal or recombinant antibody. Further, the ELISA kit may include an antibody specific to a control group protein. Additionally, the ELISA kit may include a reagent to detect an attached antibody, such as labeled secondary antibody, chromophores, enzyme (e.g., enzyme conjugated with antibody), and other substances that may bind to substrate or antibody thereof. Also, for example, the kit may be a rapid kit including essential elements necessary to perform a rapid test that shows the analysis results. The rapid kit may include an antibody specific to a protein. The antibody has high specificity and affinity to each marker protein, and almost no cross reactivity to the other proteins, and is a monoclonal, polyclonal or recombinant antibody. Further, the rapid kit may include an antibody specific to a control group protein. Other rapid test kits may include other materials required for diagnosis such as reagents capable of detecting the bound antibodies, for example, a nitro cellulose membrane to which the specific antibody and the secondary antibody are fixed, a membrane coupled with the beads bound to the antibody, the absorption pad, and the sample pad. Also, for example, the kit may be a multiple reaction monitoring (MRM) kit in the MS/MS mode, the kit including essential elements necessary to perform quantitative analysis. Selected ion monitoring (SIM) is a method that uses ions produced by bombardment on the source region of a mass spectrometer, whereas MRM is a method that uses ions obtained by selecting specific ions from broken ions and bombarding the selected ions through the source of another connected MS. Through the MRM analysis method, a protein expression level in an individual having a cancer may be compared with a protein expression level of a normal group, or the same individual, and a significant increase or decrease in the protein in the mutation gene may be verified to diagnose the onset of cancer.

According to another embodiment, provided is a method of detecting a mutation of a TERT, the method including mixing a nucleic acid template to be amplified to the PCR primer composition to prepare a mixture; performing an amplification reaction on the mixture; and detecting a mutation of a TERT from/in a product of the amplification reaction.

The amplification reaction may be performed at a temperature in a range of 80° C. to 100° C. for 5 minutes to 25 minutes. The temperature for the amplification may be in a range of 80° C. to 100° C., 80° C. to 95° C., 80° C. to 90° C., 85° C. to 100° C., 85° C. to 95° C., or 87° C. to 97° C. Here, when the temperature for the amplification is lower than these ranges, there is a problem of non-specific products being amplified, and when the temperature for the amplification is higher than these ranges, there is a problem of the amplification not occurring or a decrease in its efficiency. Also, the time for the amplification may be in a range of 5 minutes to 25 minutes, 5 minutes to 20 minutes, 5 minutes to 15 minutes, 5 minutes to 10 minutes, 10 minutes to 25 minutes, 10 minutes to 20 minutes, or 15 minutes to 25 minutes. Here, when the time for the amplification is less than these ranges, there is a problem of the amplification not occurring or a decrease in its efficiency, and when the time for the amplification is greater than these ranges, there is a problem of non-specific products being amplified.

Also, the amplification reaction may be performed for 35 cycles to 45 cycles. The amplification reaction may be performed for 35 cycles to 45 cycles, 35 cycles to 42 cycles, 35 cycles to 40 cycles, 37 cycles to 45 cycles, 37 cycles to 42 cycles, 38 cycles to 42 cycles, or 40 cycles to 45 cycles. Here, when the number of cycles of the amplification is less than these ranges, there is a problem of the amplification not occurring or a decrease in its efficiency, and when the number of cycles of the amplification is greater than these ranges, there is a problem of non-specific products being amplified.

In one embodiment, the performing of the amplification reaction may include adding betaine. The betaine reagent may be used to resolve problems such as a decrease in PCR efficiency and non-specific amplification caused by a nucleotide sequence that has a high ratio of G and C in a template DNA, that is repetitive, or that has a complex structure.

When the PCR primer composition according to an embodiment is used, there is an advantage of excellent amplification efficiency as compared to when PCR is performed using conventional primers. In particular, when PCR is performed using conventional primers, secondary PCR using secondary primers was necessary, but when the PCR primer composition according to an embodiment is used to perform PCR, amplification was possible with one PCR. Also, even damaged DNA may be amplified, and thus an accurate amplification product may be obtained, and the results may be consistent regardless of the person performing the experiment.

### Advantageous Effects of Disclosure

The present invention relates to a PCR primer composition for detecting a mutation of a telomerase reverse transcriptase (TERT), and a use thereof, wherein a TERT gene may be amplified under specific conditions by using a forward primer set represented by SEQ ID NO: 1 and a reverse primer set represented by SEQ ID NO: 2, and thus a mutation of TERT may be detected. Also, despite that a size of the amplification product is relatively large, a mutation in formalin-fixed paraffin-embedded tissues may be effectively detected.

### BRIEF DESCRIPTION OF DRAWINGS

FIG. 1 shows a gene sequence of a promoter region of telomerase reverse transcriptase (TERT), wherein the yellow high-lighted parts in the gene sequence show the positions of forward and reverse primers, which represent the 124th nucleotide, C, and the 146th nucleotide, C;

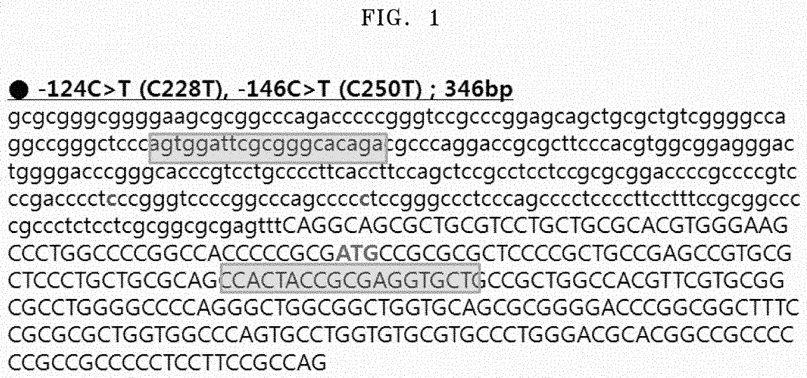

FIG. 2A shows a base sequence of a wild-type TERT gene, wherein the arrows indicates the 146th nucleotide, C, of a wild type and the 124th nucleotide, C, of a wild type;

FIG. 2B shows a first mutation sequence of a TERT gene, wherein the first mutation sequence has the 146th nucleotide, C, substituted with T;

FIG. 2C shows a second mutation sequence of a TERT gene, wherein the second mutation sequence has the 124th nucleotide, C, substituted with T;

FIG. 2D shows the results of electrophoresis on an agarose gel after amplifying mutation of TERT using primers of Example 1;

FIG. 3A shows forward and reverse primer sequences of Comparative Example 1;

FIG. 3B shows the results of TERT gene amplification performed using the primer sequences of Comparative Example 1; and

FIG. 4 shows a reverse primer sequence of Comparative Example 2.

### MODE OF DISCLOSURE

Hereinafter, the present disclosure will be described in more detail with reference to examples. The examples are for only descriptive purposes, and it will be understood by those skilled in the art that the scope of the present disclosure is not construed as being limited to the examples.

### EXAMPLE

### Example 1. Primer Design for Detection of TERT Mutation

Primers were designed to perform a PCR amplification for detection of mutation of telomerase reverse transcriptase (TERT). First, after connecting to the Primer 3 Plus (http://www.bioinformatics.nl/cgi-bin/primer3plus/primer3plus.cgi), a nucleotide sequence of TERT to be targeted was entered. Next, in General Settings, the conditions of primer size (250 bp to 400 bp), temperature (60° C.), and primer GC % (50) were entered, and “Pick Primers” was clicked. Among many results derived therefrom, primers that match the conditions of the present invention were selected.

Example 2. Verification of Detection of TERT Mutation

A mutation of TERT was detected using the primers designed in Example 1. After preparing a PCR mixture including 5×Band Doctor, DNA was amplified using a PCR machine once the PCR component was completed. The PCR reaction conditions were the same as in general PCR conditions. In particular, 7 μl of the template and 6 μl of H₂O were mixed in a PCR mixture (1 μl of each of 10 pM forward and reverse primers, 2 μl of 10×reaction buffer solution, 2 μl of 5 mM dNTP, and 1 μl of 50 U/μl Tap polymerase) to prepare a reaction solution. Then, a process of performing the PCR at 95° C. for 15 minutes, at 95° C. for 20 seconds, at 58° C. for 40 seconds, and at 72° C. for 1 minute was repeated 40 times. Upon completion of the reaction, the tube was spun down at 8,000 rpm or above for about 10 seconds to collect the solution adhering to the cap. The solution and the amplified PCR product were added to 0.2% agarose gel and were subjected to electrophoresis at 10 V.

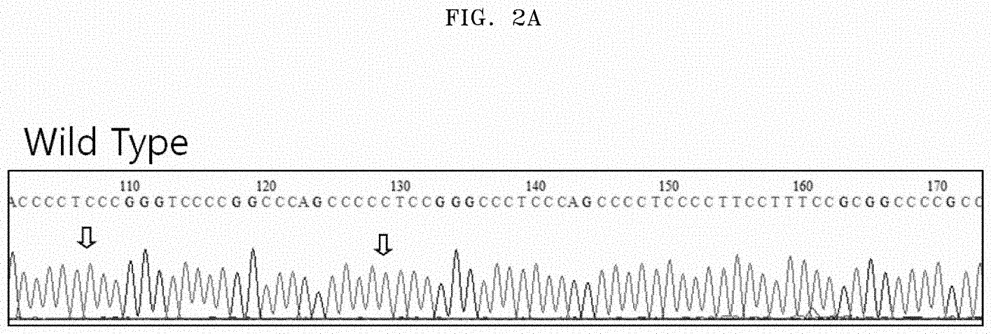

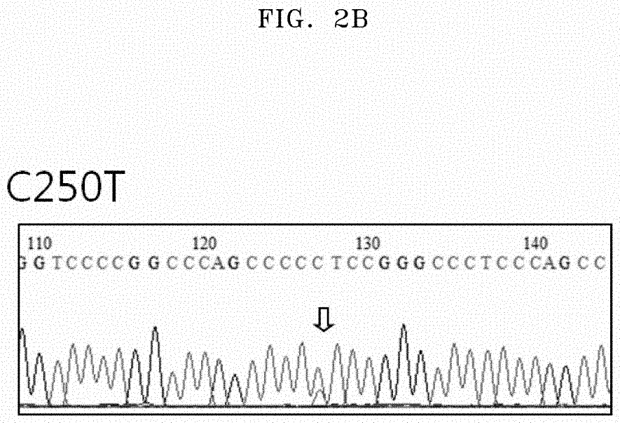

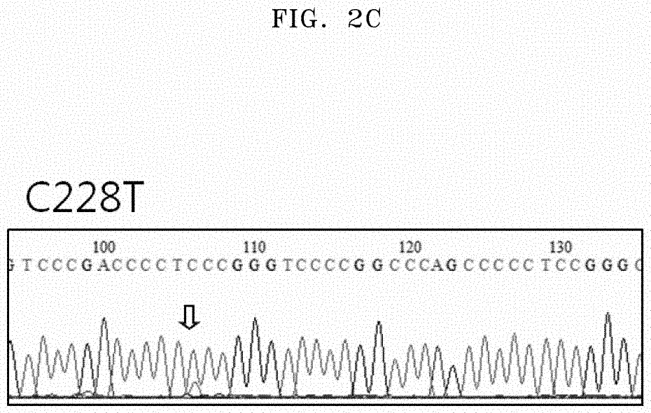

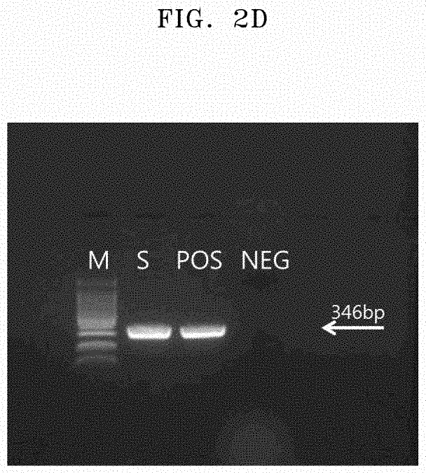

FIG. 2D shows the results of the electrophoresis on the agarose gel after amplifying mutation of TERT using the primers of Example 1. In FIG. 2D, M refers to a 100 bp ladder marker, S refers to a sample, POS refers to a positive control, and NEG refers to a negative control.

As shown in FIG. 2D, it was confirmed that the amplified PCR product was 346 bp.

### Comparative Example

Comparative Example 1. When Primers for Detecting TERT Mutation are used for Thyroid Cancer

The forward and reverse primers disclosed in the reference, Highly prevalent TERT promoter mutations in aggressive thyroid cancers (published on Sep. 24, 2013) were used to detect a TERT mutation in the same manner as in Example 2.

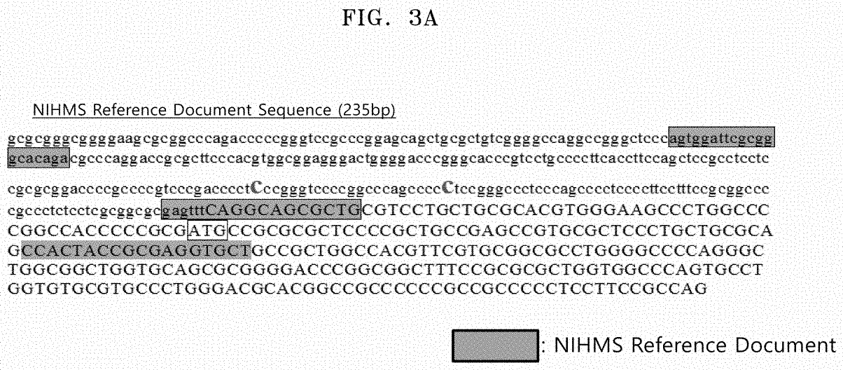

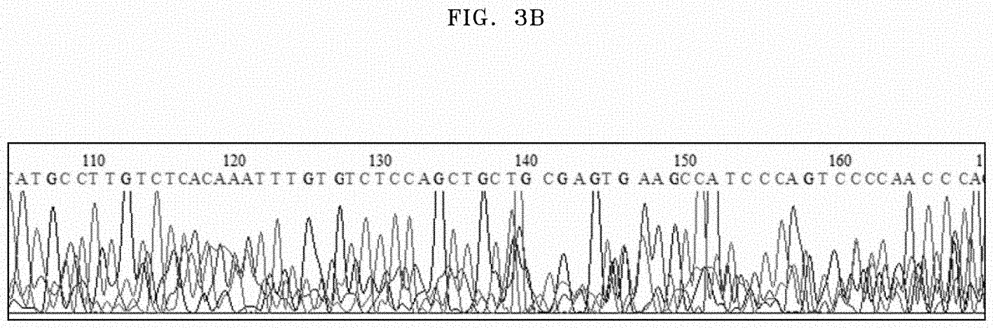

FIG. 3A shows the forward and reverse primer sequences of Comparative Example 1. The positions of the forward and reverse primers were indicated by boxes in sky-blue, and since the position of the reverse primer is very close to the target position, the target region may not be amplified when the sequencing is performed using the reverse primer in the diagnostics lab.

FIG. 3B shows the results of TERT gene amplification performed using the primer sequences of Comparative Example 1. It was confirmed that when the TERT gene was amplified using the primer sequences of Comparative Example 1, the sequencing result was not readable.

Comparative Example 2. When Primers for Detecting TERT Mutation are used for Glioma

The forward and reverse primers disclosed in the reference, TERT promoter mutations occur frequently in gliomas and a subset of tumors derived from cells with low rates of self-renewal (published on Feb. 26, 2013) were used to detect a TERT mutation in the same manner as in Example 2.

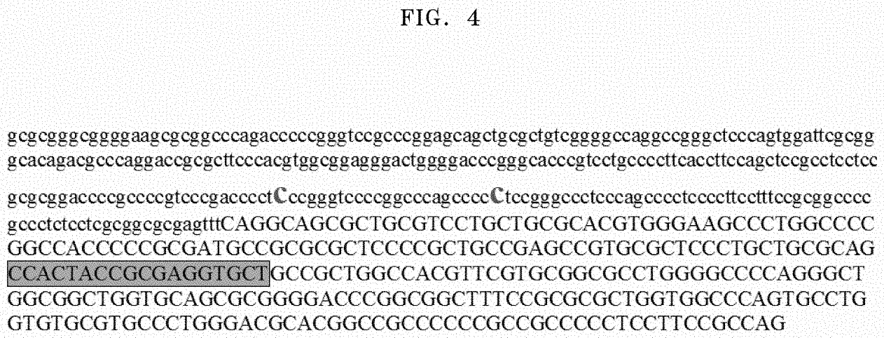

FIG. 4 shows a reverse primer sequence of Comparative Example 2. The general forward primer sequence of Comparative Example 2 has an error, where the position of the nucleotide may not be found. In this regard, a sequence that matches 100% with the forward primer of Example 1 may not be found in the nucleotide base sequence of TERT of Comparative Example 2, and thus amplification of the sequence amplified by the primer is not possible.

Comparative Example 3. When Primers of Comparative Examples 1 and 2 are Mixed

The forward primer of Comparative Example 1 and the reverse primer of Comparative Example 2 were used to detect a TERT mutation in the same manner as in Example 2.

As a result, the sequencing results were not readable as in Comparative Examples 1 and 2, nor a sequence that matches 100% with the primers of Example 1 was found.

It will be understood by those skilled in the art that the foregoing description of the present invention is for illustrative purposes only and that those of ordinary skill in the art may readily understand that various changes and modifications may be made without departing from the idea or essential characteristics of the present invention. Therefore, it should be understood that the embodiments described above are illustrative in all aspects and should not be construed as limiting the scope of the present invention.

1. A polymerase chain reaction (PCR) primer composition for a mutation of a telomerase reverse transcriptase (TERT), the PCR primer composition comprising a primer set represented by SEQ ID NO: 1 and a primer set represented by SEQ ID NO: 2.

2. The PCR primer composition of claim 1, wherein the mutation refers to substitution of T for C, which is the 124th nucleotide of a gene represented by SEQ ID NO: 3; and substitution of T for C, which is the 146th nucleotide of a gene represented by SEQ ID NO: 3.

3. The PCR primer composition of claim 1, wherein a reaction buffer solution, dNTPs, or a DNA polymerase.

4. A kit for detecting a mutation of a telomerase reverse transcriptase, the kit comprising the PCR primer composition of claim 1.

5. A method of detecting a mutation of a telomerase reverse transcriptase (TERT), the method comprising:
mixing a nucleic acid template of a TERT gene to the PCR primer composition of claim 1 to prepare a mixture;
performing an amplification reaction on the mixture; and
detecting a mutation of a TERT in a product of the amplification reaction.

6. The method of claim 5, wherein the amplification reaction is performed at a temperature in a range of about 80° C. to about 100° C. for about 5 minutes to about 25 minutes.

7. The method of claim 5, wherein the amplification reaction is performed for 35 cycles to 45 cycles.

8. The method of claim 5, wherein the performing of the amplification reaction comprises adding betaine.

9. The method of claim 5, wherein a size of the product of the amplification reaction is in a range of 330 base pairs (bp) to 400 bp.


SEQUENCE LISTING (3 sequences)

SEQ ID NO: 1 - DNA, 20 bp, Artificial
  TERT
agtggattcgcgggcacaga

SEQ ID NO: 2 - DNA, 18 bp, Artificial
  TERT
agcacctcgcggtagtgg

SEQ ID NO: 3 - DNA, 577 bp, Artificial
  TERT
gcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccag